(19) United States
(12) Patent Application Publication
(10) Pub. No.: US 2014/0148714 A1
(43) Pub. Date: May 29, 2014

(54) AUTOMATIC ONLINE DELINEATION OF A MULTI-LEAD ELECTROCARDIOGRAM BIO SIGNAL

(71) Applicants: Hossein Mamaghanian, St-Sulpice (CH); Francisco Rincon Vallejos, Madrid (ES); Nadia Khaled, Ecublens (CH); David Atienza Alonso, Ecublens (CH); Pierre Vandergheynst, Ecublens (CH)

(72) Inventors: Hossein Mamaghanian, St-Sulpice (CH); Francisco Rincon Vallejos, Madrid (ES); Nadia Khaled, Ecublens (CH); David Atienza Alonso, Ecublens (CH); Pierre Vandergheynst, Ecublens (CH)

(73) Assignee: ECOLE POLYTECHNIQUE FEDERALE DE LAUSANNE (EPFL), Lausanne (CH)

(21) Appl. No.: 13/995,249

(22) PCT Filed: Dec. 20, 2011
(86) PCT No.: PCT/IB2011/055816

§ 371 Date: Aug. 30, 2013

(30) Foreign Application Priority Data
Dec. 20, 2010 (IB) PCT IB2010 055939

(51) Int. Cl. A61B 5/04; A61B 5/044; A61B 5/00

(52) U.S. Cl. CPC A61B 5/04012; A61B 5/726; A61B 5/0006; A61B 5/0022; A61B 5/0024; A61B 5/044
USPC 600/509

(57) ABSTRACT

Method for automatic online delineation of an electrocardiogram (ECG) bio signal, said method comprising the detection of said bio signal through several leads followed by the combination of those multiple acquisitions into a single root-mean-squared (RMS) curve, said RMS curve being then undergoing a real-time single-lead delineation based on a mathematical processing.

### FIELD OF INVENTION

The present invention relates to the acquisition and monitoring of electrocardiogram (ECG) bio signals.

It more precisely relates to online (or real-time) delineation of such signals.

### STATE OF THE ART

Among the relevant cardiac signals, the noninvasive electrocardiogram (ECG) has long been used as a means to diagnose diseases reflected by disturbances of the heart's electrical activity. Beyond traditional electrocardiography, the automated processing and analysis of the ECG signal has been receiving significant attention and has witnessed substantial advances [1], [2]. In particular, a large body of algorithms have been proposed for the detection of the ECG characteristic waves, so-called ECG delineation, following a variety of approaches based on low-pass differentiation [3], the wavelet transform (WT) [4][6], dynamic time warping [7], artificial neural networks [8], hidden Markov models [9], or morphological transforms [10].

Traditionally, the automatic analysis of ECG signals, including filtering and delineation, was either taking place online on bulky, high-performance bedside cardiac monitors, or performed offline during a postprocessing stage after ambulatory ECG recording using wearable, yet obtrusive, ECG data loggers (Holter devices). Recently, however, a significant industrial and academic effort has been dedicated to online automatic ECG analysis on miniature, wearable and wireless ECG monitors as an enabler of next-generation mobile cardiology systems. These efforts essentially resulted in the development of two commercial products and a research prototype: Toumaz's Sensium Life Pebble [11], a CE-certified ultra-small and ultra-low-power monitor for single-lead ECG, heart rate (HR), physical activity, and skin temperature measurements with a reported autonomy of five days on a hearing aid battery; Corventis's PiiX [12], a CE and FDA-cleared lead-less band-aid-like ECG sensor able to perform continuous arrhythmia detection based on HR measurements; and finally IMEC's prototype of a single-lead bipolar ECG patch [13] for ambulatory HR monitoring with a claimed 10-day autonomy on a 160 mAh Li-ion battery. Accordingly, state-of-the-art unobtrusive wireless mobile/ambulatory ECG monitors are single lead and limited to embedded HR measurement and analysis.

### GENERAL DESCRIPTION OF THE INVENTION

An object of the invention is to provide an automatic online delineation of a multi-lead ECG bio signal.

Another object of the invention is to provide an embedded platform for monitoring an ECG bio signal.

Another object of the invention is to minimize the computational complexity.

Another object of the invention is to reduce the memory requirements of the stored ECG signals to fit the very tight area and memory size available in low-power embedded systems.

Another object of the invention is to minimize the energy consumption of the provided embedded platform.

All those objects are present in the invention which concerns a method for automatic online delineation of an electrocardiogram (ECG) bio signal, said method comprising the detection of said bio signal through several leads followed by the combination of those multiple acquisitions into a single root-mean-squared (RMS) curve, said RMS curve being then undergoing a real-time single-lead delineation based on a mathematical processing.

Any ECG bio signal variant (with different number of leads) of interest, in the context of ambulatory, remote and mobile health and lifestyle applications and human-machine interfaces and interactions, can be monitored and delineated in the context of the invention. In a preferred embodiment of the invention, when the ECG signal is acquired, the first step performed is to remove the baseline wander (mainly caused by respiration, electrode impedance changes due to perspiration and body movements) in each of the leads, since the quality of the subsequent delineation depends on the baseline wander correction. The following two algorithms may be used to perform this task.
Cubic Spline Baseline Estimation. This method uses a third-order polynomial to approximate the baseline wander, which is then subtracted from the original signal. To do so, a representative sample (or knot) is chosen for each beat from the silent isoelectric line, which is represented by the PQ segment in most heart rhythms. The polynomial is then fitted by requiring it to pass through successive triplets of knots.
Morphological Filtering. This method applies several erosion and dilation operations to the original ECG signal to estimate the baseline wander. It first applies an erosion followed by a dilation, which removes peaks in the signal. Then, the resultant waveforms with pits are removed by a dilation followed by an erosion. The final result is an estimate of the baseline drift. The correction of the baseline is then done by subtracting this estimate from the original signal.

Of course, any other suitable algorithm for performing this task may be used.

Once all the leads are filtered, they are combined using a root mean squared (RMS) approach into a multi-lead signal, which provides an overall view of the cardiac phenomena and is independent of the lead system used.

Then, a single-lead delineation is performed on the RMS curve generated after the combination of all the leads. Any appropriate algorithm can be used to perform this delineation step, in particular:
Wavelet Transform (WT). This method performs the detection of all characteristic points (onset, peak, and end) of the ECG waves using preferably a quadratic spline WT, which produces derivatives of smoothed versions of the input ECG signal at five dyadic scales (i.e., 2¹ to 2⁵). The choice of these scales is based on the observation that most of the energy of the ECG signals lies within these scales. In particular, it has been shown that the energy of the QRS complex is lower in scales higher than 2⁴, and that the P and T waves have significant components at scale 2⁵.
According to this WT-based ECG delineation principle, the WT at scale 2^{k} is proportional to the derivative of the filtered version of the input ECG signal with a smoothing function at scale 2^{k}. Then, the zero crossings of the WT correspond to the maxima or minima of the smoothed ECG signal at different scales, and the maximum absolute values of the WT are associated with maximum slopes in the smoothed ECG signal. Moreover, each sharp change in the input ECG signal is associated with a line of maxima or minima across the scales. Accordingly, using this information of local maxima, minima, and zero crossings at different scales, the WT-based algorithm identifies the fiducial points of the ECG signal.
Multiscale Morphological Derivative (MMD). This approach is also based on the fact that all the singular points of the ECG signal (onset, peak and end of the QRS complex and P and T waves) correspond to maxima and minima of the signal. Therefore, a singular point is defined as a point where derivatives on the left and right exist with different signs.
Advantageously, the MMD is applied on the original signal and the delineation of the fiducial points of the ECG signal is performed only taking into account the transformed signal. This delineation detects the local minima and maxima of the transformed signal, since, as aforementioned, the MMD transform converts the singular points of the original ECG signal into local maxima and minima.

The results generated after the delineation are then preferably sent to a Wireless Body Sensor Network (WBSN) coordinator/sink. Optionally, the raw ECG signal can also be sent to the WBSN coordinator. In this case, Compressed Sensing (CS) may be advantageously used to compress the original raw ECG signals and therefore reduce airtime over energy-hungry wireless links. This CS-based compression algorithm consists of three processing stages. In the first one, a linear transformation based on sparse binary sensing is applied to the original ECG signal. The input data is simply multiplied by a sparse binary random matrix in which each column has a very small number d of nonzero entries equal to 1 (more details can be found in [14]), where d is chosen depending on the sparsity of the input signal. The use of a fixed binary sensing matrix, combined with the quasi-periodic nature of the ECG signal, yields to very similar consecutive measurement vectors. Then, interpacket redundancy removal is performed to compute the difference between consecutive vectors, therefore, only this difference is further processed. Since encoding the difference needs less bits than encoding the original samples, 3 bits can be saved (considering an input signal encoded with 12 bits). Thus, interpacket redundancy removal adds 25% of compression due to this reduction in the bit depth without losing the original information (loss-less compression). In the last stage, Huffman coding is preferably applied to encode the compressed signal to be wirelessly transmitted.

### DETAILED DESCRIPTION OF THE INVENTION

The invention will be better understood with the following non-limiting example which relates to the evaluation of a real-time multi-lead Wavelet Transform (WT) and Multiscale Morphological Derivative (MMD)-based electrocardiogram (ECG) wave delineation and filtering algorithms, which were ported and optimized to a state-of-the-art commercial wearable embedded sensor platform.

A typical use of this system in clinical practice is the 3-lead configuration in ambulatory ECG monitoring. The 3 leads are simultaneously acquired at a sampling frequency of 250 Hz and then filtered to remove the baseline wander. In this case the cubic spline baseline estimation approach is used. According to the previous general description of this technique, as “knot” is selected a point within the PR segment (the time interval between the end of the P wave and the beginning of the QRS complex). More specifically, the point that is 28 ms (seven samples) is experimentally chosen before the beginning of the QRS complex. Consequently, detecting a “knot” boils down to detecting the beginning of the QRS complex, using a simplified version of the WT-based single-lead delineator. Then, once three knots are detected, these points are used to fit a third-order polynomial, which provides an approximation of the baseline wander. This approximation is further subtracted from the original signal.

Once the 3 leads xₗ[n], with l=1, 2, 3, are filtered, they are combined in a single multi-lead signal x_{RMS}[n] according to the following equation:

$${x}_{RMS} [ n ] = \sqrt{\frac{1}{3} {\sum }_{l = 1}^{3} {x}_{l}^{2} [ n ]}$$

where n denotes the discrete-time index.

The resultant signal x_{RMS}[n] is then delineated using the WT or MMD-based algorithms mentioned above. In both cases, after obtaining the derivatives of the signal, the algorithm looks for maxima and minima in the transformed signal, which corresponds with the fiducial points of the original ECG wave. The first point to be detected is the R peak, since it is the most clear and easy to detect. Then, the algorithm delineates the secondary waves around it, namely, the onset and end of the QRS complex. Finally, the algorithm detects the boundaries and peaks of the P and T waves.

All the delineation results are sent to a coordinator, such as a mobile phone, where the results are displayed and stored. In addition, the raw ECG signal is also sent to the coordinator, using Compressed Sensing and 70% compression ratio, which leads to a good signal recovery.

As mentioned previously, the invention is not limited to the use of WT or MMD-based algorithms.

The same applies to the filtering algorithms.

Any suitable algorithm can be used.

### PRIOR ART REFERENCES CITED IN THE DESCRIPTION

[1] L. Sörnmo and P. Laguna, “Bioelectrical Signal Processing in Cardiac and Neurological Applications”, Amsterdam, The Netherlands: Elsevier Academic Press, 2005, ch. 7.
[2] U. R. Acharya, J. S. Suri, J. A. E. Spaan, and S. M. Krishnan, “Advances in Cardiac Signal Processing”, New York: Springer-Verlag, 2010, ch. 2-4.
[3] P. Laguna, R. Jane, and P. Caminal, “Automatic detection of wave boundaries in multilead ECG signals: Validation with the CSE database”, Com put. Biomed. Res., vol. 27, no. 1, pp. 45-60, February 1994.
[4] C. Li, C. Zheng, and C. Tai, “Detection of ECG characteristic points using wavelet transforms”, IEEE Trans. Biomed. Eng., vol. 42, no. 1, pp. 21-28, January 1995.
[5] J. S. Sahambi, S. Tandon, and R. K. P. Bhatt, “Using wavelet transform for ECG characterization”, IEEE Eng. Med. Biol., vol. 16, no. 1, pp. 77-83, 1997.
[6] J. P. Martinez et al., “A wavelet-based ECG delineator: evaluation on standard databases”, IEEE Trans. Biomed. Eng., vol. 51, no. 4, pp. 570-581, April 2004.
[7] H. Vullings, M. Verhaegen, and H. Verbruggen, “Automated ECG segmentation with dynamic time warping”, in Proc. IEEE EMBC, 1998, pp. 163-166.
[8] Z. Dokur, T. Olmez, E. Yazgan, and O. Ersoy, “Detection of ECG waveforms by neural networks”, Med. Eng. Phys., vol. 19, no. 8, pp. 738-741, 1997.
[9] S. Graja and J. M. Boucher, “Hidden Markov tree model applied to ECG delineation”, IEEE Trans. Instrum. Meas., vol. 54, no. 6, pp. 2163-2168, 2005.
[10] Y. Sun, K. L. Chan, and S. M. Krishan, “Characteristic wave detection in ECG signal using morphological transform”, BMC Cardiovasc. Disorders, vol. 5, no. 28, 2005.
[11] Toumaz Technology. (2009). [Online]. Available: http://www.toumaz.com/public/news.php?id=92
[12] Corventis, 2009. [Online]. Available: http://www.corventis.com/AP/nuvant.asp
[13] R. F. Yazicioglu, T. Torfs, J. Penders, I. Romero, H. Kim, P. Merken, B. Gyselinckx, H. J. Hoo, and C. V. Hoof, “Ultra-low-power wearable biopotential sensor nodes”, in Proc. IEEE EMBC, September 2009.
[14] H. Mamaghanian, N. Khaled, D. Atienza Alonso and P. Vandergheynst. “Compressed Sensing for Real-Time Energy-Efficient ECG Compression on Wireless Body Sensor Nodes”, in IEEE Transactions on Biomedical Engineering, vol. 58, num. 9, p. 2456-2466, 2011.

1. Method for automatic online delineation of an electrocardiogram (ECG) bio signal, said method comprising the detection of an ECG bio signal through several leads followed by the combination of those multiple acquisitions into a single root-mean-squared (RMS) curve, said RMS curve being then undergoing a real-time single-lead delineation based on a mathematical processing.

2. Method according to claim 1 wherein said real-time single-lead delineation is based on a multi-scale Wavelet transform.

3. Method according to claim 1 wherein said real-time single-lead delineation is based on a multi-scale morphological Derivative.

4. Method according to claim 1 comprising the removal of baseline wander on each of the leads before the generation of the RMS curve.

5. Method according to claim 4 wherein the removal of baseline wander includes a morphological filtering.

6. Method according to claim 4 wherein the removal of baseline wander includes a cubic spline baseline estimation.

7. Method according to claim 1 comprising the automatic online delineation of the most relevant waves of an ECG, namely QRS, P & T.

8. Method according to claim 1 wherein Compressed Sensing (CS) is simultaneously applied.

9. Wireless Body Sensor Network (WBSN) for monitoring a bio signal according to claim 1.

10. WBSN according to claim 9 comprising a standard mobile or wearable embedded platform such as an iPhone for displaying said bio signal.
